(19) United States
(12) Patent Application Publication
(10) Pub. No.: US 2022/0138672 A1
(43) Pub. Date: May 5, 2022

(54) METHOD AND SYSTEM FOR CARRYING OUT A STOCKTAKE ON A STOCK OF OBJECTS, AS WELL AS A READER, A SERVER AND COMPUTER READABLE MEDIUM

(71) Applicant: Nedap N.V., Groenlo (NL)

(72) Inventors: Danny Jacobus Theodorus HAAK, Enschede (NL); Barry HAANSTRA, Veenendaal (NL); Ron WEIKAMP, Winterswijk (NL); Dennis Wilhelmus MULLER, Enschede (NL)

(21) Appl. No.: 17/299,855

(22) PCT Filed: Apr. 5, 2019
(86) PCT No.: PCT/NL2019/050207

§ 371 Date: Jun. 4, 2021

(30) Foreign Application Priority Data
Dec. 5, 2018 (NL) 2022135

(51) Int. Cl. G06Q 10/08; G06K 7/10; G06K 19/07; H04B 17/318

(52) U.S. Cl. CPC G06Q 10/087; H04B 17/318; G06K 19/0723; G06K 7/10099

(57) ABSTRACT

The invention relates to a method for carrying out a stocktake on a stock of objects, the objects being distributedly located in a multiplicity of spaces, the spaces being physically separated from each other, and each of the objects being provided with a tag which is configured for generating a wireless identification signal associated with the tag. Further, the invention relates to a system, a reader device, a computer program product, and a server.

### FIELD OF THE INVENTION

The present invention relates to a method for carrying out a stocktake on a stock of objects, the objects being located distributedly in a multiplicity of spaces, the spaces being physically separated from each other, and each of the objects being provided with a tag which is configured for generating a wireless identification signal associated with the tag. Furthermore, the invention relates to system, a reader, and a server for carrying out such a method. Also, the invention relates to computer program products comprising instruction sets for carrying out the above method.

### BACKGROUND

Radio frequency identification (RFID), the sending of (high) frequency identification signals by means of tags and labels, is used for, inter alia, making a stock count for the purpose of stock management. Articles in a shop are then, for example, provided with RFID tags, and a count can be carried out with a portable reader with which the identification signals can be received.

For obtaining a proper and useful count, it is often desired that a distinction can be made between articles that are in different spaces. For example, it may be of interest to make a count of articles that are on different floors of a shop, or to make a distinction between articles that are in the stock or on the sales floor.

One of the advantages of RFID is that the technology is capable of reading tags over greater distances. A disadvantage involved here, however, is that the signals from RFID tags can also be received through walls, so that a count of articles within a single space may become inaccurate. The location of particular articles can no longer be properly established in this way.

The solution that is used to resolve such problems is for spaces to be mutually shielded, so that signals in one space cannot be received in the other space anymore. This shielding may be done, for example, by cladding the wall with aluminum foil, or by applying paint with conductive particles. This is a rather costly method, in particular in shops, to prevent the RFID signals being detected in the wrong space. Moreover, an additional disadvantage is that in this way also the reception of other wireless signals is counteracted, so that, in this way, also Wi-Fi, Bluetooth, 4G, DECT and other communication signals cannot be received in all spaces anymore.

### SUMMARY OF THE INVENTION

It is an object of the present invention to counteract the above disadvantages of the state of the art, and to enable a stock count for the purpose of stock management to be performed on the basis of RFID, whereby a distinction can be made between articles that are in different spaces.

To this end, the invention provides according to a first aspect thereof a method for carrying out a stocktake on a stock of objects, the objects being located distributedly in a multiplicity of spaces, the spaces being physically separated from each other, and each of the objects being provided with a tag which is configured for generating a wireless identification signal associated with the tag, wherein the method comprises carrying out in each space of the multiplicity of spaces the steps of:
a. receiving with a reader one or more identification signals associated with one or more tags; and
b. for each of the one or more received identification signals, recording signal strength information in a signal strength record, the signal strength information being indicative of a reception strength of the respective identification signal as received by the reader, and wherein the signal strength information is furthermore linked to space information indicative of the space in which the reader has received the identification signal; and
   wherein, following the carrying out of the steps a. and b. in each space of the multiplicity of spaces, the method further comprises the steps of, for each tag of the multiplicity of tags:
c. determining, for the respective tag, on the basis of the signal strength records for all spaces of the multiplicity of spaces, of the signal strength record that indicates the greatest reception strength; and
d. storing, for the respective tag, the space information linked to the signal strength record determined in step c.

The method according to the invention makes it possible to establish directly from the received identification signals that have been received with a reader device, in what space the respective tags are. Here, use is made of, inter alia, the fact that identification signals from tags that are in the immediate surroundings of the reader are received more strongly than the signals from tags at a greater distance. Tags that are in the same space as the reader are, in many cases, at a smaller distance than tags in other spaces. Furthermore, the invention makes use of the difference in signal attenuation between signals that, on the one hand, are received directly through the air and, on the other hand, have to go through a physical separation before being received. The signal attenuation caused by a wall, for example, is much greater, so that signals that are received from a tag that, with respect to the reader, is on the other side of a wall, are significantly weaker than when the reader and the tag are in the same space. Because of these two effects, the space in which the tag whose identification signals were received is present, can be reliably established on the basis of the signal strengths.

Suppose, for example, that an identification signal from a particular tag is measurable in three different spaces of a shop (once in the space in which the tag is present and once in each of the two other spaces). In accordance with the invention, in each of the three spaces physically separated from each other, a measurement will be made, and thus in each of these spaces the identification signal of the above tag is received. The received identification signals are recorded together with the signal strength information of the received signal, and in a manner in which it is later derivable in what space what measurement was made. After all measurements have been carried out, several records will be available for the tag of this example: in any case one record per space in which measuring was done. By comparing the records that are available for this tag, it can be established that the signal strength of the received signal in one of the measurements—the measurement in the space in which the tag is actually present—is significantly greater than the signal strengths in the two other records. On the basis of this comparison, it can therefore be established with the method according to the invention where the tag is.

The method therefore makes automatic detection of the space possible, by comparing the received signal strengths of the signals of each tag with each other. A stock management count can therefore, with the aid of the method according to the invention, be carried out rapidly and reliably and provides direct insight into where the articles or objects are. Thus, for example, a user can establish directly whether the stock that is available on the sales floor in his shop needs to be supplemented, and whether this can be done from the stock stored in the stock space or that reordering is to be done. The stock management system can thus be kept up-to-date in a simple manner, because such a count can be carried out in each space of the shop, if so desired every day or several times a day.

The spaces physically separated from each other can be different spaces in a building, but may also be spaces that are physically separated from each other in a different manner. For example, the method may also be carried out with different mutually separate cupboard spaces or containers provided with container walls. Also possible is a mixed form of these, for example by counting in a sales space and in a stock space in which different cupboards, containers, a cold store and other partial spaces are present.

According to an embodiment, the method furthermore comprises a step of: e. on the basis of the space information stored for each tag in step d., for each space of the plurality of spaces, generating a count of all tags that have been linked to the respective space, the count being indicative of an amount of the objects that are located in the respective space. In this manner, a whole stock administration can be kept up-to-date in a simple manner.

According to an embodiment, the method comprises, for recording the signal strength information in the signal strength record in step b., the step of: sending of the signal strength information by the reader to a server, with the signal strength record being generated by the server; or the reader generating the signal strength record for each tag and storing the same in a memory unit. In other words, recording can take place centrally in the memory of a server or locally in a memory of a reader device. In the latter case, the reader device, at the end of a measurement, or after several measurements in several spaces, can itself carry out the steps c. and d. or send the whole record file to a server. Also, it is possible that a reader device receives records from other reader devices and processes them in an own local file, or that all records of all reader devices are centrally processed by a server.

According to a specific embodiment, the method comprises the step of sending of the signal strength information by the reader to the server, the method further comprising the step of: prior to the sending of the signal strength information, causing it to be established by the reader for each of the one or more received identification signals whether the identification signal is associated with a tag in respect of which already at least one signal strength recording has taken place, wherein the signal strength information of the respective received identification signal is only sent to the server when the reception strength of the received identification signal is greater than the reception strength of the identification signal associated with the signal strength record for the tag. By having such establishing done by the reader, the number of signals to be sent to the server can be strongly limited, so that the reader saves energy and so that the chance of failure issues in the system due to interference between transmitted signals diminishes. A reader device then presents identification signals for recording only when the signal strength is greater than as indicated in the signal strength record. Only in that case does recording of the signal strength information add anything to the records already available. This way, moreover, the whole measurement becomes more reliable in that temporary signal attenuations due to unforeseeable circumstances (for example, the transmitted signal being temporarily weaker due to a person just happening to be present between the source and the receiver) are filtered out.

According to an embodiment, for providing the space information, the method comprises the step of, for each space in which the steps a. and b. are carried out, causing the space information to be inputted by a user. Alternatively, it is also possible, according to other embodiments, that the method comprises a step of determining of the space information in each space by the reader on the basis of at least one of: a positioning system, detection of Wi-Fi signals associated with the respective space, or triangulation of beacon signals from a multiplicity of transmitters based on time of flight of the beacon signals. In other words, detection of the space in which measuring is being done can take place automatically, but it is also possible that the space information is entered manually.

According to yet another alternative embodiment of the invention, one or more spaces of the multiplicity of spaces are provided with a reference tag, the reference tag being configured for generating a wireless reference signal associated with the reference tag, and wherein the method comprises: receiving, by the reader, of one or more reference signals; determining, by the reader, of signal strength information for each of the one or more reference signals; and identifying, from the signal strength information of the received reference signals, one reference signal having the greatest signal strength, for determining the space which the reader is in, for providing the space information. In effect, reference tags in this embodiment are detected similarly to the tags of the objects to be counted, the difference being that the reference tags are recognizable as reference tags whose location in the system is fixed. In a special embodiment the reference tag may actually be furnished by the tag of one of the objects in respect of which it is known in what space the object is present. For example, of a tag of an object in the display window of the shop, it can be certain that it is in the same space as the tags of all objects in the sales space (because, for instance, the display window is in the sales space). That is why the signal of this tag can also be used to establish that measuring is being done in the sales space.

According to an embodiment of the method according to the invention, the steps a. and b. are carried out by a multiplicity of readers, and for carrying out steps c. and d. the signal strength records are sent by one or more of the readers, for combining of the signal strength records either by a server or by at least one other one of the multiplicity of readers. This embodiment, in which the measurements in the different spaces are carried out with different readers, has already been briefly discussed hereinabove. The method can be carried out by a group of users, for example in buildings of a larger size where a team of workers cooperate in different spaces of the building to make the count.

In the above embodiments, according to particular variants of these embodiments, the method can comprise: determining, by each reader of the multiplicity of readers, on the basis of all identification signals received by the reader, of a greatest reception strength; and dividing each of the reception strengths of all identification signals received by the reader, by the determined greatest reception strength, for normalizing the signal strength information for each reader. By dividing the signal strengths established by each of the readers, by the signal strength of the very strongest identification signal that has been received by the reader concerned, the received signals from different readers can be reliably compared with each other. After all, the reception strength of a received signal may be distorted by, for example, a weak battery of the reader or a defect on the receiver of the reader, or by an external source of interference such as a high humidity of the air in one of the spaces. Normalizing the signal strengths in this manner ensures that the relative strengths with respect to the strongest signal are compared with each other, so that such inaccuracies have no influence on the end result.

According to an embodiment, the method comprises carrying out in each space of the multiplicity of spaces the steps of: following step a. or b., establishing by the reader of one of the following parameters: a direction, with respect to the reader, from which the respective identification signal has been transmitted; a reception point of time of the identification signal; and a time of flight of a transmission of the identification signal from the tag to the reader. Establishing such parameters provides additional information about the signal, with which the reliability or functionality can be increased. By, for example, establishing a direction from which a signal is being transmitted, it is also possible to trace an object within a space. Also, this information can be used to establish with greater certainty that an object, for example, is in a particular partial space. The time of flight provides insight into the distance between the reader and the object, which information is also applicable for the purpose of tracing objects or increasing reliability.

According to an embodiment, the tag comprises a radio frequency identification (RFID) circuit. The invention is particularly suitable to be operative on the basis of RFID signals. RFID signals are already being used on a large scale in shops and storage spaces to enable objects to be electronically distinguished from each other. Furthermore, the signal strength information can comprise a received signal strength indicator or make use thereof. According to an embodiment, the method comprises, for each received identification signal, identifying the tag, for identifying either the object or a type of object. Furthermore, according to an embodiment, the reader is at least one element from a group comprising: a portable reader device; a fixed reader device which is in one of the multiplicity of spaces; and a portable computer provided with a computer application for carrying out the method, such as a mobile phone or tablet computer. Because the use of mobile phones, in particular smartphones, is widespread and these devices are typically provided with the necessary technology to be able to receive RFID signals, these devices can be used to perform a count within a method according to the invention.

According to a second aspect, the invention provides a system for carrying out a stocktake on a stock of objects, the objects being located distributedly in a multiplicity of spaces, the spaces being physically separated from each other, and each of the objects being provided with a tag which is configured for generating a wireless identification signal associated with the tag; the system comprising a server, at least one reader device, and a communication network, wherein the server is operatively connected with the communication network, and wherein the reader device is provided with communication means for exchanging data signals via the communication network with the server, wherein the communication means comprise a receiver for receiving one or more identification signals associated with one or more tags, and wherein the reader device is provided with a processing unit for, for each of the one or more received identification signals, generating signal strength information, the signal strength information being indicative of a reception strength of the respective identification signal as received by the reader device; wherein at least one of the reader device or the server is configured for: recording the signal strength information for the respective identification signal in a signal strength record, wherein the signal strength information is furthermore linked to space information indicative of a space in which the reader device has received the identification signal; and wherein at least one of the reader device or the server is furthermore configured for, for each tag of the multiplicity of tags: determining, for the respective tag, on the basis of the signal strength records for all spaces of the multiplicity of spaces, of the signal strength record that indicates the greatest reception strength; and storing, for the respective tag, of the space information which is linked to the particular signal strength record that indicates the greatest reception strength.

According to a third aspect, the invention provides a reader device for use in a system according to the second aspect, or for use in a method according to the first aspect.

According to a fourth aspect, the invention provides a server for use in a system according to the second aspect, or for use in a method according to the first aspect.

Furthermore, according to a fifth aspect, the invention provides a computer readable medium provided with computer program product comprising instructions which, when loaded into a memory of a reader device, make the reader device suitable for carrying out a method according to the first aspect.

Furthermore according to a fifth aspect, the invention provides a computer readable medium provided with computer program product comprising instructions which, when loaded into a memory of a server, make the server suitable for carrying out a method according to the first aspect.

### BRIEF DESCRIPTION OF THE FIGURES

The invention will be discussed hereinbelow on the basis of specific embodiments thereof not intended as limiting, with reference to the appended figures, in which:

FIG. 1 is a schematic representation of a shop in which a method for carrying out a stocktake according to an embodiment of the invention is applied;

FIG. 2 shows a system according to an embodiment of the invention;

FIG. 3 shows a system according to a further embodiment of the invention;

FIG. 4 shows a system according to further embodiments of the invention;

FIG. 5 shows a system according to a further embodiment of the invention;

FIG. 6 shows a system according to a further embodiment of the invention;

FIG. 7 schematically shows a working principle of an embodiment according to the invention;

FIG. 8 is a screenshot of a count screen in a method and system according to an embodiment of the invention;

FIG. 9 is a screenshot of a count screen in a method and system according to an embodiment of the invention; and

FIG. 10 shows a method according to an embodiment of the invention.

### DETAILED DESCRIPTION

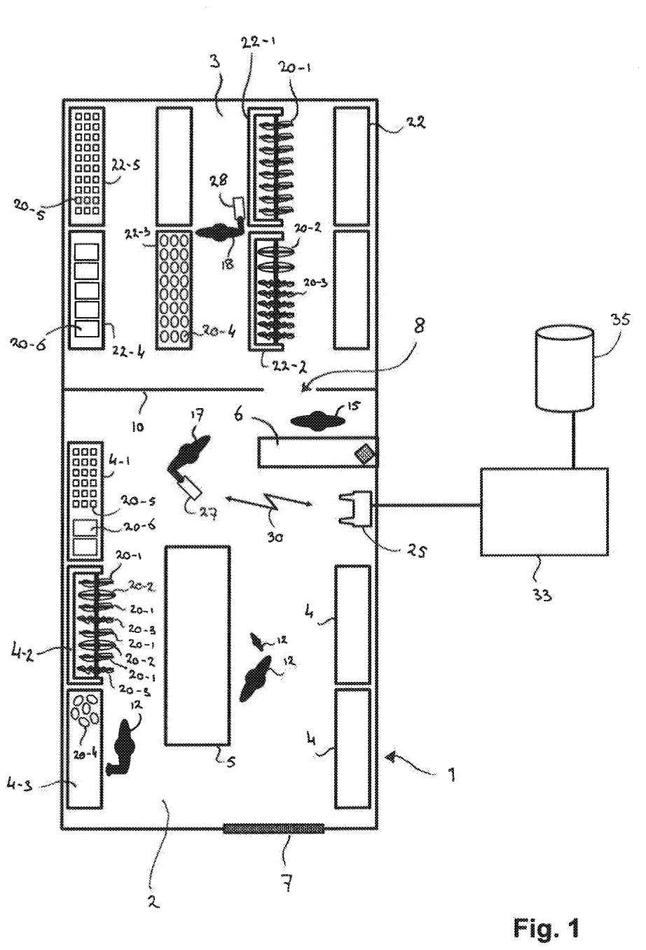

In FIG. 1, schematically, a shop 1 is shown in which a stock management method according to an embodiment of the invention is applied. The shop 1 consists of a sales space 2, which forms the real physical shop. Also, behind the sales space 2 is a stock storage 3 which is accessible via an opening 8 behind a counter 6. The sales space 2 and the stock storage 3 are physically separated by a partition 10. The partition 10 may be made of, for example, stone, plaster, wood, concrete, or any other customary construction material. The shop 1, and in particular the sales space 2, is accessible via a door 7 which is on the street side.

In the sales space 2 are a multiplicity of rack cupboards 4 and a display table 5. The rack cupboards 4 and the display table 5 show a multiplicity of sales items or articles 20 to the shoppers 12. Also in the stock management space 3, there are a multiplicity of cupboards 22, containing the stock of articles 20. In FIG. 1 the articles are generally designated with reference numeral 20, and different kinds of articles are designated with a sub-classification, for example 20-1, 20-2, 20-3, 20-4, 20-5 and 20-6. The same numbering of articles can be found in the stock space 3. For clarity, in FIG. 1 only a part of the articles are represented. Furthermore, also the cupboards 4 and 22 are designated with a sub-classification to indicate specific cupboards. The sales space 2 shows, for example, rack cupboards 4, but in particular also rack cupboards 4-1, 4-2 and 4-3. Also in the stock space 3, rack cupboards 22 are shown, but also, in particular, the cupboards 22-1, 22-2, 22-3, 22-4 and 22-5.

The shop staff includes a sales assistant 17, a cashier 15, and a stock management assistant 18. The sales assistant 17 and the stock management assistant 18, in the example shown in FIG. 1, have a portable reader 27 and 28 at their disposal. The readers 27 and 28 can communicate wirelessly via a Wi-Fi network which is provided by a router 25. Wireless communication between portable reader 27 and router 25 is schematically designated with double-headed arrow 30 in FIG. 1. Coupled to the local area network (LAN) of the shop is also a server 33 which is used as stock server, and a data storage 35. The server 33, for that matter, need not necessarily be connected via a local area network, but, depending on the embodiment, may also be operatively connected in a different manner. For example, the server may be connected via a wide area network such as the Internet, or may be accessible via a mobile telecommunication network. Possible options in this regard also include a connection through power line communication (PLC) or a wireless connection via Bluetooth, or optical communication. Accordingly, the invention is not limited to a particular form of data communication or a particular type of network.

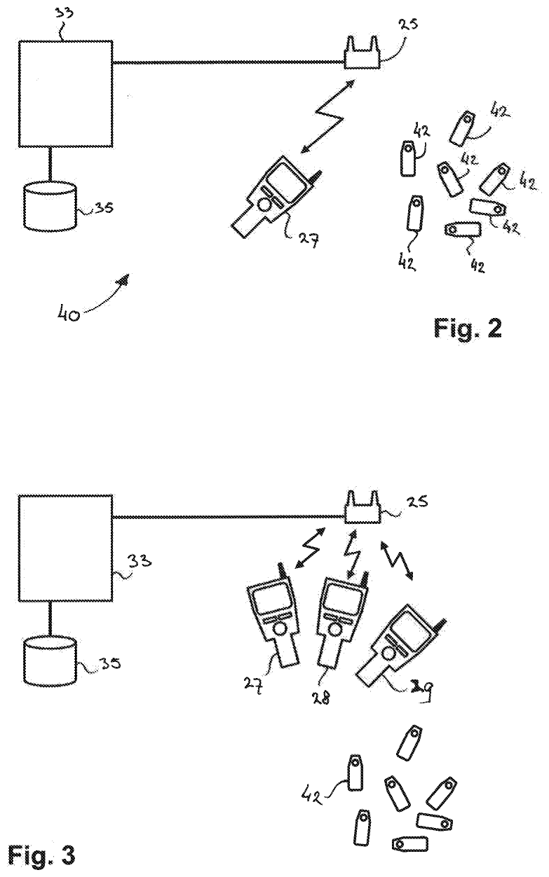

The method according to an embodiment according to the invention may be carried out in the shop as shown in FIG. 1, for instance as follows. Each of the articles 20 is provided with a radio frequency identification (RFID) tag. The RFID tags are not shown in FIG. 1, but schematic examples thereof are visible in, for instance, FIGS. 2 through 6. The RFID tags can be of the passive, active or hybrid type. An active RFID tag contains a battery and may, for instance, actively transmit an identification signal with a particular time frequency. A passive RFID tag receives an interrogation signal, for example from a reader 27, and uses energy from the interrogation signal for sending out an identification signal. In a hybrid RFID tag there is also a battery, but the RFID tag will, for instance, merely send out an identification signal when it receives an interrogation signal from, for example, reader 27. The method according to the invention can work with each of the above types of RFID technologies, i.e., both the active and hybrid types, and the passive RFID tag types.

Starting from active RFID tags, the stock in the shop 1 in FIG. 1 can be counted in accordance with an embodiment of the invention, for instance as follows. The sales assistant 17 who has RFID reader 27 at hand starts a counting action, and then receives the RFID identification signals from a multiplicity of articles 20. Because each of the articles 20 is provided with a tag, and the identification signals per tag are unique for that tag, the reader 27 is able to mutually distinguish the signals from each of the articles present in the sales space. However, because the sales assistant 17 is relatively close to the stock space 3, there is a reasonable chance that the identification signals from (at least a part of) the articles in the stock space 3 are also received by the reader 27. Of each received identification signal, the reader 27 will determine the reception strength of the signal. This is expressed in a received signal strength indicator (RSSI) value. The stronger the signal, the greater the RSSI value. The reader 27 will record the signal strength information, in particular the RSSI value, of the identification signals of each tag. Also, when a particular identification signal is received several times, the reader 27 can make several records of the identification signal, or optionally the reader 27 can determine which of the received identification signals for the tag concerned was strongest, and then record this strongest value. The signal strength record made by reader 27 will be linked to space information. The space information to which the signal strength record is to be linked, depends on the space where the reader 27 is present at that moment. In some embodiments, the sales assistant 17 can input this space information manually. Other embodiments, however, include a possibility of automatically (for example on the basis of the Wi-Fi signal) establishing in which space the reader 27 is present during counting. Linking of the space information can be done per record, so that each signal strength record comprises not only the RSSI value (or a value depending thereon), but also an indicator which indicates the space. It is also possible that linking of the signal strength records to space information is done, for example, for a number of records at the same time: for example, for the sales space 2 where the reader 27 is present at this moment, a separate file may be created with signal strength records that have been made in that space during counting.

The execution of a count by the reader 27 is continued until all articles have been counted. For example, the reader 27 may continue to count until within a defined period of time no new identification signals have been recognized anymore. In another embodiment, counting is compared with, for example, information present in the computer system 33 regarding the stock. The shopkeeper knows approximately how many articles he has in stock and can use this estimate of the current stock to establish whether the stock has been counted completely.

The working principle of the present invention is based on the fact that the partition wall 10 between the stock space 3 and the sales space 2 brings signal attenuation with it. The tags of the articles that are in the stock space 3 send out identification signals that can be received on the other side of the wall 10 by the sales assistant 17. However, because these identification signals are received through the wall 10, and maybe partly via reflection through the opening 8, the reception strength of these identification signals is less than when the same identification signals are received by the reader device 28 of stock management assistant 18, since reader device 28 receives the identification signals directly through the air in the stock space 3, whereas reader 27 receives them through the wall 10. In accordance with the invention, however, both reader 27 and reader 28 will continue to count and record those identification signals. This means that the reader 27 counts not only the articles that are in the cupboards 4-1, 4-2, and 4-3, but also the articles from which identification signals are received but which are in the stock space 3. The same thing happens in reader device 28, which also counts, at least in part, the articles in the sales space 2. Between the readers 27 and 28, therefore, double counts take place. The method according to the present invention, however, provides a reliable and correct count of the stock in that of each received identification signal the signal strength information is stored in a signal strength record. In this manner, then, afterwards, after the reader devices 27 and 28 have finished counting, the identification signals can be distinguished from each other by their reception strength. For example, in cupboard 4-1 article 20-5 is present. Reader device 27 receives an identification signal from the tag of article 20-5 and stores the signal strength information of the identification signal in a signal strength record. In the stock space 3, in cupboard 22-5, also a multiplicity of articles 20-5 are present. The identification signal of each of the articles 20-5 is unique, so that the articles in their totality can be counted on the basis of their identification signals. However, by also taking the signal strength record of each identification signal 20-5 into account, the reader 27 can establish whether the identification signals of the articles 20-5 that are in cupboard 22-5 in the stock space 3 are in the same space as article 20-5 in cupboard 4-1 in the sales space. The recording unit 27 will establish that the signals originating from articles in rack cupboard 4-1 in the sales space are appreciably stronger than the signals of the similar articles 20-5 in cupboard 22-5 in the stock space 3. According to the invention, the reader 27 in an embodiment thereof is intended to count merely the articles that are in the same space—the sales space 2. Due to the signals from the articles in cupboard 22-5 being appreciably weaker, reader 27 will recognize the signals 20-5 from rack cupboard 4-1 as being in the same space 2 as where the reader 27 is. The signal strength records of the identification signals will therefore be linked to the space information of sales space 2 and be stored, for example in the memory 35 of the computer system. The reader 28 carries out a similar count and establishes that the articles 20-5 in rack cupboard 22-5 are in the same space—the stock space 3—as the reader 28. The space information of stock space 3 is accordingly linked to the signal strength records of the articles 20-5 in the cupboard 22-5, and thus stored in the memory 35 of the computer system 33. Also the reader device 28 carries out a count until all objects have been counted: so, until received identification signals contain no new unique identification signal anymore within a defined period of time.

The records that have been made with the reader devices 27 and 28 are now to be combined and analyzed by the computer system 33. As has already been indicated above, these records will include double counts. For example, article 20-5 from rack cupboard 4-1 will have been counted by both reader 27 and reader 28, and a signal strength record will therefore be available in the memory 35 in both the count of reader 28 and the count of reader 27. By combining the information of all counts, it can be determined on the basis of the signal strength in which of the spaces 2 or 3 what articles are present. For example, the signal strength record of article 20-5 in rack cupboard 4-1 will have been stronger in the count of recording unit 27 than in the count of recording unit 28. Both signal strength records occur in the total set of signal strength records, but are distinguishable from each other on the basis of their signal strength. According to the invention, the computer system 33 will determine on the basis of the signal strength records which one of the signal strength records made for the tag 20-5 in rack cupboard 4-1 indicates the highest reception strength. The signal strength of reader 27 is then clearly the winner of the two, and because it is known that reader 27 has carried out a count in the sales space 2, the computer system 33 can establish that article 20-5 is in the sales space 2. In this way, with the method according to the invention, an exact count can be made of what the stock of articles is in the stock space 3 and in the sales space 2. This count can be done fully automatically, and afterwards it can be established what articles are on the shelves of the sales space 2, and what articles are in the stock space 3. To give an example, during the counting of the articles 20, also the clothing in clothes rack 4-2 has been counted. Hanging in clothes rack 4-2 are different kinds of clothing, unsorted, for example trousers 20-1, jackets 20-2 and blouses 20-3. The sales assistant 17 makes a count with the reader 27 and asks the stock management assistant 18 to do the same in stock space 3. The signal strength records done with all readers 27 and 28 are combined by computer system 33 and subsequently analyzed. On the basis of the signal strength records it can be established that in the sales space 2 there are four pairs of trousers 20-1. It can also be seen that in rack 4-2 there are two jackets 20-2 hanging, and that further there are two blouses 20-3 in the sales space 2. At the same time, it can be established that in the stock space 3 there are eight pairs of trousers 20-1, two jackets 20-2 and six blouses 20-3. The total stock of clothes in the shop therefore comprises twelve pairs of trousers, four of which in the sales space and eight of which in the stock space; four jackets 20-2, two of which in the sales space and two in the stock space; and eight blouses 20-3, two of which in the sales space and six in the stock space. It can also be seen that, of article 20-4, six specimens are in the sales space 2 and twenty-four specimens are in the stock. Of article 20-6, two specimens are present in the sales space, and five more are in the stock space. Of article 20-5, effectively, eighteen specimens are available in the sales space, and thirty more in the stock space. As has been described hereinabove, with the method according to the invention, in a relatively simple manner, a stock management count can be carried out with a reliable result. It has been explained hereinabove that computer system 33 establishes on the basis of the signal strength records whether articles are in the same space as the reader 27. By comparing the counts of all readers with each other, it is possible to establish for each received identification signal in which space of the building this article is present. Reliability can even be optionally enhanced by not only establishing within which count the strongest identification signal has been received by the reader, but also confirming this information by verifying whether the signal strength of the same identification signal was appreciably weaker for the other readers. For instance, the identification signal of article 20-5 in rack cupboard 4-1 in the sales space 2 will have come in relatively strong for reader 27, but at the same time will have come in appreciably weaker for reader 28. Identification may already be done by comparing the signal strength record of reader 27 with that of reader 28, and thus determining that the signal strength record of reader 27 was the stronger and therefore probably indicates the space that this article was in. Confirmation is possible by establishing on the basis of the much weaker signal of the signal strength record of reader 28 that this article was probably not in the stock space. The reliability of this count is thereby enhanced still further.

The method according to embodiments of the present invention can be carried out with different embodiments of systems according to the present invention.

In FIG. 2 a first embodiment is shown of a system according to the present invention. The system 40 comprises a central server 33 with a data storage memory 35, for example a hard disk. Central server 33 is coupled to a router 25, in a manner similar to that shown in FIG. 1. However, the system has a single reader device 27 with which the whole method is carried out. This embodiment in FIG. 2 therefore does not involve a plurality of readers as in FIG. 1, but involves a single reader 27 with which all counts are carried out. The method according to the invention as carried out with a system 40 as shown in FIG. 2 differs to some extent from the above-explained method in FIG. 1. With the system 40, all counts are to be performed with the same reader 27. This means that when the sales assistant 17 executes a count in the shop space 2 in FIG. 1, another count is to be carried out in turn in a stock management room 3 with the same reader 27. For each of these counts it is to be indicated in which space it has been carried out. For example, the sales assistant 17 starts a count and indicates in reader 27 that the count is carried out in sales space 2. As soon as the count has been done, the sales assistant 17 gives the reader 27 to stock management assistant 18, who indicates in the stock space 3 that the reader 27 is now in stock space 3. Thereupon, stock management assistant 18 likewise starts a count for the stock space 3. Both counts are passed on to computer system 33 of system 40 and the records are stored in the memory 35 as indicated hereinbefore. In FIG. 2, also the tags 42 are shown which can be attached to the articles 20.

FIG. 3 shows an alternative embodiment of a system 43 according to an embodiment of the invention. The system 43 is effectively equal to the system as shown in FIG. 1, in which a plurality of readers 27, 28, and 29 can be used for concurrently counting in a plurality of spaces. The signals are sent to a router 25 which passes the records on to a central computer 33, after which they are stored in the memory 35.

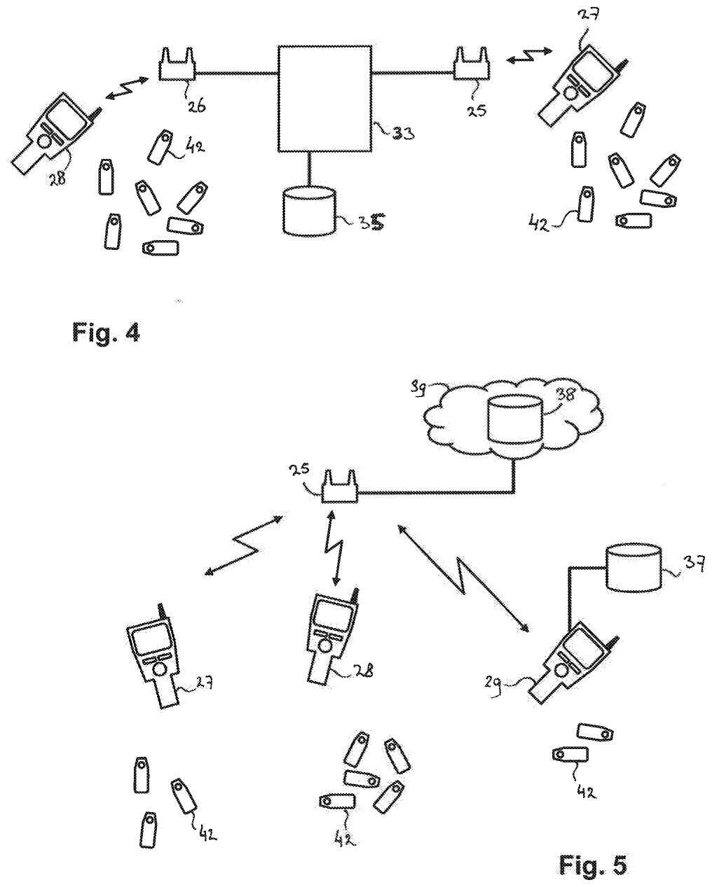

FIG. 4 shows yet another embodiment of a system 44 according to the present invention. In system 44 a central server 33 with a memory 35 is coupled to a plurality of routers 25 and 26. Each router is in a different space. The reader 27 contacts router 25 and passes signal strength records of the tags 42 on via router 25 to computer system 33. Establishing the space information in order for this information to be linked to signal strength records can now be done in different ways. In a first embodiment, each of the readers 27 and 28 can establish, on the basis of the received Wi-Fi signal from the routers 25 and 26, which router 25 or 26 the reader makes contact with. Based on this, for example, reader 27 can establish that it is in the sales space 2 and reader 28 can establish that it makes contact with a router 26 which is present, for example, in the stock space. Another possibility, according to another embodiment, is that the computer system 33 establishes via which router 25 or 26 the signal strength records of the readers 27 and 28 are received. The signal strength records of reader 28 are received via router 26 and the signal strength records of reader 27 are received via router 25. Because the routers are in different spaces 2 and 3 of the shop, it can be established on the basis of the router signal where the readers 27 and 28 are. According to yet another possibility, the system 44 includes more than two routers, and triangulation is possible of the received router signals, or of the received signals of the readers 27 and 28. Based on such a triangulation, also the position of the reader 27 or 28 can be established, and the information regarding the space in which the reader 27 or 28 is present can be found.

In yet another embodiment, shown in FIG. 5, there is no central server 33 present, but the different readers 27, 28 and 29 send their data signals via router 25 to each other. For example in this way, the readers 27 and 28 can forward their signal strength records and possibly the space information to the reader 29 which is provided with a memory 37. The memory 37 in this case is in the reader 29, and all signal strength records are stored in the memory 37. Alternatively, as also shown in FIG. 5, a memory 38 may be present in the cloud 39, and is accessible via the Internet. The signal strength records of all readers 27, 28 and 29 can then be sent, for example, directly to the memory 38.

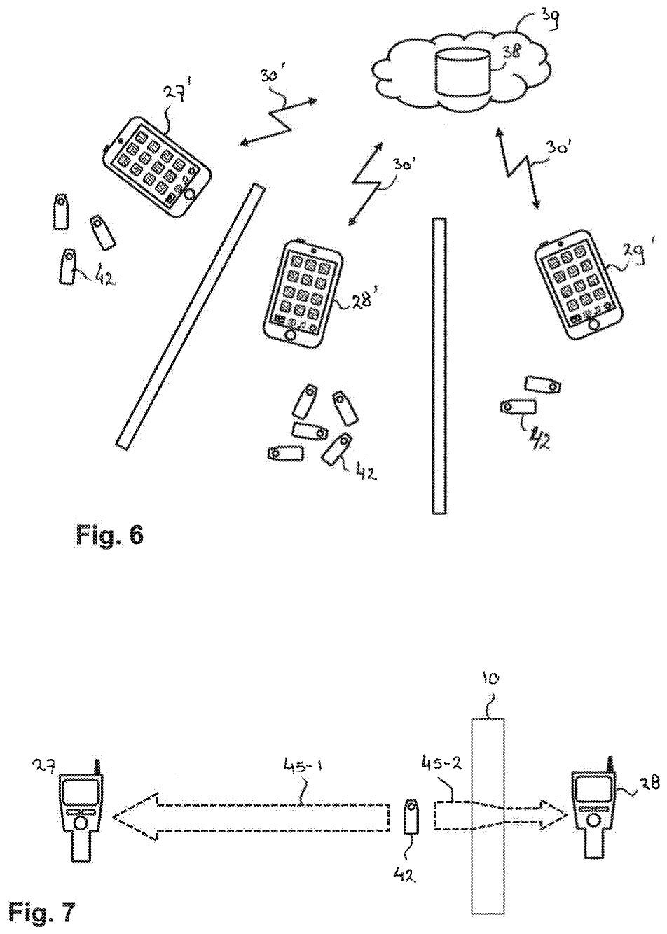

In yet another embodiment, shown in FIG. 6, no use is made of special dedicated readers 27, 28 and 29, but the stock management method is carried out with the aid of smartphones 27′, 28′ and 29′ which fulfill the functions of the readers 27, 28 and 29. The system 47 can for example communicate via a GSM network, and the wireless signals 30′ are GSM signals. Each of the phones 27′, 28′ and 29′ sends its signal strength records via these wireless signals 30′ to a storage medium 38 in the network 39. The operation of the method according to the invention is otherwise identical to what has been described above.

FIG. 7 shows the working principle on which the present invention is partly based. In FIG. 7 tag 42 is shown which is directly behind a wall 10. On the other side of the wall is a reader 28, and in the same space there is also a reader 27 at a somewhat greater distance from the tag 42. The tag 42 sends an identification signal 45 which is received by both readers 27 and 28. However, the partial signal 45-1 which is received by reader 27 is received directly through the air and is hardly attenuated. By contrast, the partial signal 45-2 which is received by the reader 28 is significantly attenuated because it has been received through the partition wall 10. This is schematically indicated in FIG. 7 with the arrow being narrower on the other side of the wall 10 on the side of the reader 28 than on the side of the tag 42. This attenuation makes it possible to analyze the received signals on the different readers 27 and 28 and to establish where the tag 42 is with respect to the wall 10.

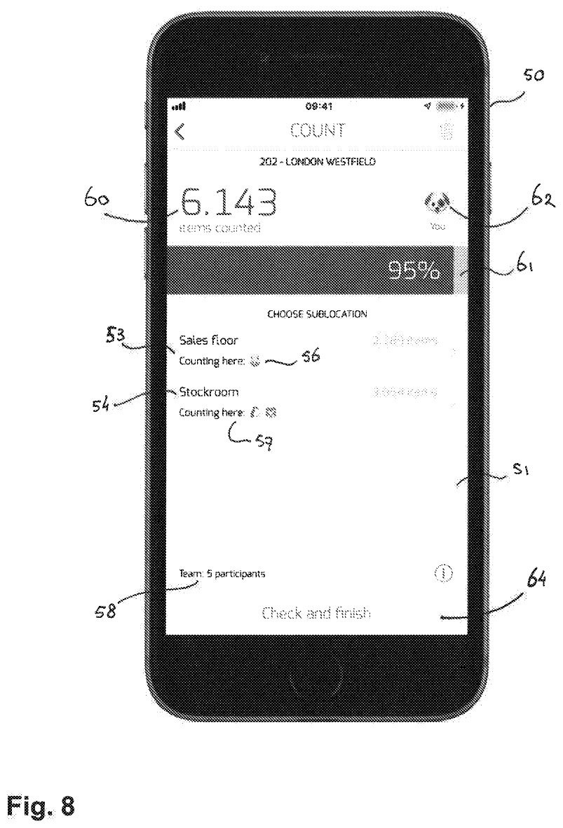

FIG. 8 shows a screenshot of a telephone 50 with which a count is being carried out. FIG. 8 shows the count screen 51 and demonstrates that user 62 is carrying out a count and has already counted 6143 items (indicator 60). Also shown in the screen is a relative progress indicator 61, indicating (by estimation) what amount of the total of available identification signals has already been counted. Also visible in the screen is what other assistants are engaged in the count. On the sales floor (partial screen 53) there is an assistant (as represented by indicator 56) who has already counted 2189 articles. In the stockroom (partial screen 54), as represented by indicator 57, two assistants are present who together have counted 3954 articles. In total, the team for counting the stock consists of five participants, as represented by indicator 58. The fifth team member is possibly offline and is therefore not visible in the count screen of FIG. 8. The button ‘Check and finish’ 64 provides to the user 62 the possibility to stop counting and finish, and, possibly, to count again.

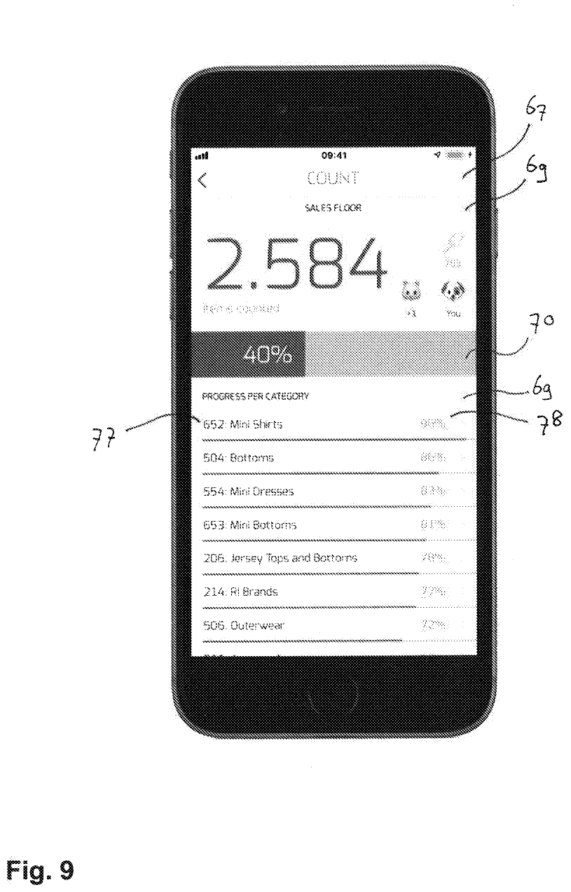

FIG. 9 is a screenshot of a count screen 67 of the stock management method, for displaying an overview for a specific location during counting. The progress indicator 70 indicates what percentage of the stock (i.e., the available identification signals) in the space concerned has already been counted. The location indicator 69 indicates for what location the count is being carried out. In FIG. 9, the count on the sales floor is performed jointly by two team members, who together have counted 2584 items. According to progress indicator 70, this is 40% of the total number of items. Count screen 67 also includes partial screen 69 which reflects progress per category of articles. On the basis of the identification signals, however, it can also be established for each counted item what (type of) article is involved. Thus, it is known from counting that, for example, 652 mini shirts have been counted, as indicated by indicator 77. Because during initialization in a similar way an estimate of the total number of mini shirts can be made, it can be established that the indicated number involves 96% of the total stock of mini shirts, as indicated by indicator 78. Instead of making estimates on the basis of unique identification signals, as explained hereinabove, in an alternative embodiment also an estimate of the total number of items can be obtained from already available information in the administration system. Thus, for example, the information from a previous count may be used, or on the basis of the administration of deliveries a total stock of articles present (possibly broken down by kind of article) can be determined.

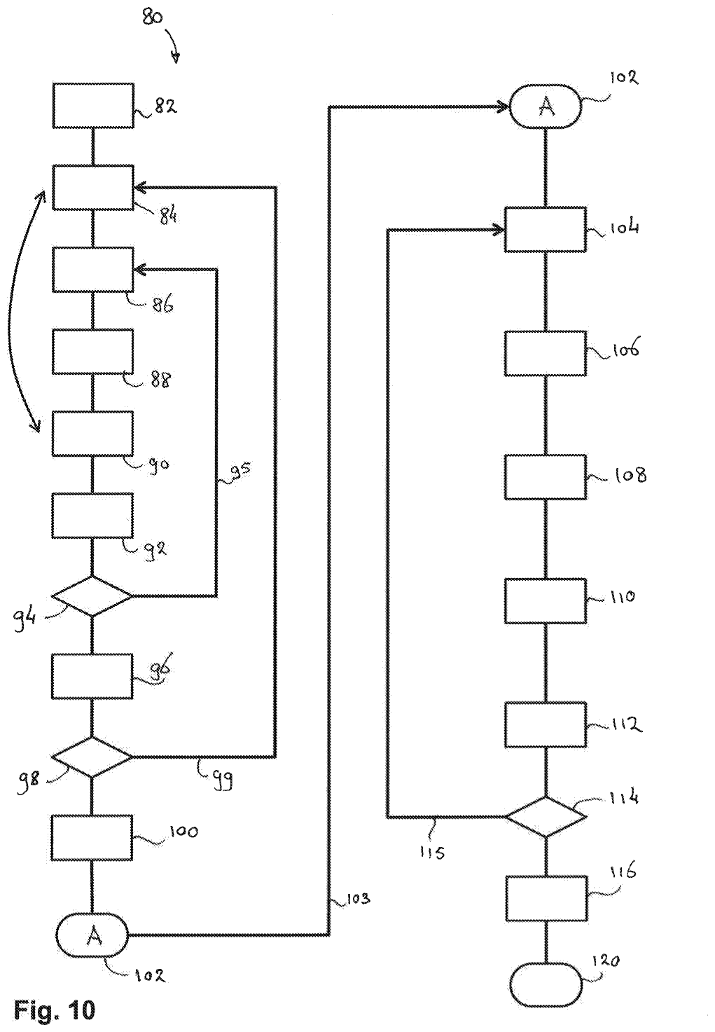

FIG. 10 shows an embodiment of a method according to the present invention. The method 80 starts in step 82 with an initialization of the reader. For example, the reader can establish on the basis of all received identification signals how strong the strongest identification signal is that is received. This information will be used later to normalize the signal strength of the identification signals from the different tags. In step 84, the reader detects which space it is in. This can be done in various manners, for example through entry by a user or through an automatic determination of the space (for example based on the Wi-Fi signal, Wi-Fi positioning system, a global positioning system (GPS) or in a different manner). In step 86 the reader receives the identification signals from a multiplicity of tags. For each received identification signal, it is determined in step 88 what the signal strength is, for example by expressing it in a received signal strength indicator value (RSSI value). Next, in step 90, the signal strength information is linked to the space information which has been found in step 84. Optionally, the RSSI value found is normalized in step 88 by dividing it by the strongest signal strength found in step 82 during initialization. By normalizing the value of the received signal strength on the basis of the signal strength of the most strongly received signal, the advantage is achieved that the signal strength records that have been obtained with different readers can more easily be compared with each other. Influential factors such as, for example, the battery status of the reader itself or any defects, can be filtered out in this way. After the linking in step 90, the signal strength record is stored in step 92. This can be done in the reader or in a central server 33. In step 94 it is established whether any new identification signal is yet to be recorded. When this is the case, the method proceeds via loop 95 with step 86, receiving of the identification signal. When, however, all identification signals have been processed, and the reader establishes, for example, that for a particular period (five seconds) no new identification signals are received anymore, it can be established in step 94 that no new identification signal is to be processed and the method continues in step 96. In step 96, it is established that the complete space has been counted. Next, it is determined in step 98 whether any other space is to be counted with the same reader. When this is the case, the method goes back via loop 99 to step 84 and counting is carried out in another space. When, however, no new space is to be counted, the method proceeds to step 100. In step 100, the collected signal strength records of all readers and all counts are combined in one set. Via A (102), the method then goes on in step 104 where analyzing of the measuring data is commenced. In step 104, it is established for a tag, within the set of signal strength records, how many signal strength records have been stored. When, for example, counting has been done with four readers, it may well be that an identification signal has been received on each of the readers and so occurs four times in the set. However, the signal strength of each signal is different. In step 106 an inventory is made of the available signal strength records for the respective tag in the set. Then, from the available signal strength records, it is determined in step 108 which of the signal strength records indicates the strongest signal strength. On the basis of this determination, it can be determined in step 110 in which space this signal has been counted. Next, in step 112 it is established that the respective tag is in the space found, and this information is stored. In step 114 it is then determined whether the method should analyze a next tag, and when this is the case, the method proceeds via loop 115 to step 104. When, however, all tags have been analyzed, and there is a complete overview of the stock, and of where the articles are, the method goes on in step 116 where the analysis is finished and the analyzed data is released or presented. Thereupon, in step 120, the method according to the invention. is ended.

The above-described specific embodiments of the invention are intended for illustration of the principle of the invention. The invention is only limited by the appended claims.

1. A method for carrying out a stocktake on a stock of objects, the objects being distributedly located in a multiplicity of spaces, the spaces being physically separated from each other, and each of the objects being provided with a tag that is configured for generating a wireless identification signal associated with the tag,
wherein the method comprises carrying out, in each space of the multiplicity of spaces, the steps of:
a. receiving with a reader one or more identification signals associated with one or more tags; and
b. recording, for each of the one or more received identification signals, signal strength information in a signal strength record, the signal strength information being indicative of a reception strength of the respective identification signal as received by the reader, and wherein the signal strength information is furthermore linked to space information indicative of a space in which the reader has received the identification signal; and
wherein, following the carrying out of the steps a. and b. in each space of the multiplicity of spaces, the method further comprises performing the steps of, for each respective tag of the multiplicity of tags:
c. determining, for the respective tag, on the basis of the signal strength records for all spaces of the multiplicity of spaces, the signal strength record that indicates the greatest reception strength; and
d. storing, for the respective tag, the space information that is linked to the signal strength record determined in step c.

2. The method according to claim 1, further comprising a step of:
e. generating, on the basis of the space information stored for each tag in step d, for each respective space of the plurality of spaces, a count of all tags linked to the respective space, the count for each respective space being indicative of an amount of the objects that are located in the respective space.

3. The method according to claim 1, wherein for recording the signal strength information in the signal strength record in step b., the method comprises the step of:
sending the signal strength information by the reader to a server, with the signal strength record being generated by the server; or
the reader generating the signal strength record for each tag and storing the same in a memory unit.

4. The method according to claim 3, comprising the step of the sending of the signal strength information by the reader to the server, wherein the method further comprises the step of:
prior to the sending the signal strength information, causing to be established by the reader, for each of the one or more received identification signals, whether the identification signal is associated with a tag for which at least one signal strength recording has already taken place, wherein the signal strength information of the respective received identification signal is only sent to the server in accordance with determining the reception strength of the received identification signal is greater than the reception strength of the identification signal associated with the signal strength record for the tag.

5. The method according to claim 1, wherein for providing the space information the method comprises the step of, for each space in which the steps a. and b. are carried out, causing the space information to be inputted by a user.

6. The method according to claim 1, further comprising a step of determining, of the space information in each space, by the reader on the basis of at least one of: a positioning system, detection of Wi-Fi signals associated with the respective space, or triangulation of beacon signals from a multiplicity of transmitters based on time of flight of the beacon signals.

7. The method according to claim 1, wherein one or more spaces of the multiplicity of spaces is provided with a reference tag, the reference tag being configured for generating a wireless reference signal associated with the reference tag, and wherein the method comprises:
receiving, by the reader, of one or more reference signals;
determining, by the reader, signal strength information for each of the one or more reference signals; and
identifying from the signal strength information of the received reference signals one reference signal having greatest signal strength, for determining the space the reader is in, for providing the space information.

8. The method according to claim 7, wherein the reference tag is furnished by the tag of one of the objects of which it is known which space the object is in.

9. The method according to claim 1, wherein the steps a. and b. are carried out by a multiplicity of readers, and wherein for carrying out steps c. and d. the signal strength records are sent by one or more of the readers, for combining of the signal strength records either by a server or by at least one other one of the multiplicity of readers.

10. The method according to claim 9, further comprising:
determining, by each reader of the multiplicity of readers, on the basis of all identification signals received by the reader, of a greatest reception strength; and
dividing each of the reception strengths of all identification signals received by the reader by the determined greatest reception strength, for normalizing the signal strength information for each reader.

11. The method according to claim 1, wherein the method comprises carrying out in each space of the multiplicity of spaces the steps of:
establishing by the reader, following step a. or b., at least one parameter taken from the group of parameters consisting of:
a direction, with respect to the reader, from which the respective identification signal has been transmitted;
a reception point of time of the identification signal; and
a time of flight of a transmission of the identification signal from the tag to the reader.

12. The method according to claim 1, wherein the tag comprises a radio frequency identification (RFID) circuit.

13. The method according to claim 1, wherein the signal strength information comprises a received signal strength indicator.

14. The method according to claim 1, wherein the multiplicity of spaces comprise at least one space type taken from the group consisting of: a building space within a building, a cupboard space, a container, and a crate or box for storage or transport.

15. The method according to claim 1, further comprising:
identifying, for each received identification signal, the tag, for identifying either the object or a type of object.

16. The method according to claim 1, wherein the reader is at least one element taken from the group consisting of:
a portable reader device;
a fixed reader device which is in one of the multiplicity of spaces; and
a portable computer provided with a computer application for carrying out the method.

17. The method according to claim 1, wherein the spaces are physically separated from each other by a physical separation.

18. The method according to claim 17, wherein the physically separated spaces are formed by different:
spaces in a building; or
mutually separate cupboard spaces or containers provided with container walls, or a cold store.

19. A system for carrying out a stocktake on a stock of objects, the objects being distributedly located in a multiplicity of spaces, the spaces being physically separated from each other, and each of the objects being provided with a tag that is configured for generating a wireless identification signal associated with the tag, the system comprising;
a server;
at least one reader device; and
a communication network,
wherein the server is operatively connected with the communication network,
wherein the reader device includes a communication interface for exchanging data signals via the communication network with the server,
wherein the communication interface comprises a receiver for receiving one or more identification signals associated with one or more tags, and
wherein the reader device is provided with a processor for generating, for each of the one or more received identification signals, signal strength information, the signal strength information being indicative of a reception strength of the respective identification signal as received by the reader device;
wherein at least one of the reader device or the server is configured for:
recording the signal strength information, for the respective identification signal, in a signal strength record,
wherein the signal strength information is linked to space information indicative of a space in which the reader device received the respective identification signal; and
wherein at least one of the reader device or the server is configured for performing, for each tag of the multiplicity of tags, the following:
determining, for the respective tag, on the basis of the signal strength records for all spaces of the multiplicity of spaces, the signal strength record that indicates the greatest reception strength; and
storing, for the respective tag, of the space information that is linked to the determined signal strength record that indicates the greatest reception strength.

20. The system according to claim 19, wherein at least one of the reader device or the server is furthermore configured for:
generating, on the basis of the space information stored for each tag, for each respective space of the multiplicity of spaces, a count of all tags linked to the respective space, the count for each respective space being indicative of an amount of the objects that are located in the respective space.

21. The system according to claim 19, wherein the reader device is configured for:
establishing, for each of the one or more received identification signals, whether the identification signal is associated with a tag for which at least one signal strength recording has already taken place, and
sending the signal strength information to the server in accordance with determining the reception strength of the received identification signal is greater than the reception strength of the identification signal associated with the signal strength record for the tag.

22. The system according to claim 19, wherein the reader device is configured to receive space information input by a user.

23. The system according to claim 19, wherein the system is provided with a multiplicity of reader devices.

24. The system according to claim 19, wherein the tag comprises a radio frequency identification (RFID) circuit.

25. The system according to claim 19, wherein the at least one reader device belongs to a group comprising: a portable reader device; a fixed reader device which is in one of the multiplicity of spaces; and a portable computer provided with a computer application for carrying out the method, such as a mobile phone or tablet computer.

26. A reader device configured for use in a system to carry out a method according to claim 1.

27. A server configured for use in a system to carry out a method according to claim 1.

28. A computer readable medium provided with computer program product comprising instructions which, when loaded into a memory of a reader device, make the reader device suitable for carrying out a method according to claim 1.

29. A computer readable medium provided with computer program product comprising instructions which, when loaded into a memory of a server, make the server suitable for carrying out a method according to claim 1.
